(19) United States
(12) Patent Application Publication
(10) Pub. No.: US 2014/0255963 A1
(43) Pub. Date: Sep. 11, 2014

(54) Degradable Detergents

(71) Applicant: THE BOARD OF TRUSTEES OF THE LELAND STANFORD JUNIOR UNIVERSITY, Palo Alto, CA (US)

(72) Inventor: Anthony Le, San Jose, CA (US)

(21) Appl. No.: 14/236,137

(22) PCT Filed: Jun. 26, 2012
(86) PCT No.: PCT/US2012/044184

§ 371 Date: May 19, 2014

Related U.S. Application Data
(60) Provisional application No. 61/524,673, filed on Aug. 17, 2011.

(51) Int. Cl. G01N 33/68; C12Q 1/44; C12Q 1/34; C07C 233/13

(52) U.S. Cl. CPC G01N 33/6848; C07C 233/13; C12Q 1/44; C12Q 1/34
USPC 435/19; 564/154; 436/86; 435/18

(57) ABSTRACT

Methods and materials relate to degradable detergents. The degradable detergents have degradable linkages that are cleaved when subjected to elevated temperature and/or reduced pressure. The detergents are compatible with spectrometric analysis, such as mass spectrometry. The surfactant comprises at least one fluorinated alkyl moiety and at least one cleavable moiety, wherein the surfactant degrades into a plurality of volatile degradation products when injected into a mass spectrometer.

### CROSS-REFERENCE TO RELATED APPLICATION

This application claims priority to U.S. provisional application Ser. No. 61/524,673, filed Aug. 17, 2011, which application is incorporated herein by reference in its entirety.

### INTRODUCTION

Detergents play a major role in a variety of applications. In proteomics, for example, detergents are used to aid in isolating and purifying proteins. Detergents are also important in monitoring and understanding enzymatic reactions upon proteins.

Mass spectrometry is a common tool for analyzing biological compounds, including analysis of proteins. Standard mass spectrometry and the related technique of matrix assisted laser desorption ionization (MALDI) provide valuable information on chemical structure. Such information can be useful in deducing protein function and conformation, and monitoring enzymatic reactions. In this context, detergents play an important role in purifying biological samples prior to analysis by mass spectrometry and other analytical methods.

Typical detergents are highly incompatible with mass spectrometers. When typical detergents are present in a sample in an appreciable amount, and when the sample is injected into the mass spectrometer, the detergent is likely to cause significant troubles. Such troubles range from total ion suppression of the individual sample to contamination of the ion source and vacuum system thereby affecting future samples. In view of this, it is common practice to remove detergents from samples prior to analysis by mass spectrometry. In many cases, the removal process is difficult and tedious, and may also result in a significant loss of sample mass.

### SUMMARY

Herein are described methods and materials relating to degradable detergents. For example, the degradable detergents have degradable linkages that are cleaved when subjected to elevated temperature and/or reduced pressure. In some aspects the degradable detergents described herein are compatible with spectrometric analysis, such as mass spectrometry.

In some aspects, herein is described a surfactant comprising at least one fluorinated alkyl moiety and at least one cleavable moiety, wherein the surfactant degrades into a plurality of volatile degradation products when injected into a mass spectrometer.

In another aspect, there is provided a surfactant comprising a compound prepared from reaction between a volatile acid and a diamine.

In yet another aspect, there is provide a surfactant comprising an adduct of a first component and a second component, wherein the first component is a fluorinated acid and the second component is an amino thiol.

In yet another aspect, there is provided a compound comprising the addition product of two cysteamines and two carboxylic acids.

In another aspect, there is provided a method for forming a surfactant, the method comprising reacting a fluorinated acid with a bifunctional linking compound to form a cleavable surfactant product.

In yet another aspect, there is provided a method for preparing an analyte, the method comprising combining the analyte with a fluorinated acid and a basic compound, wherein the fluorinated acid and basic compound react to form a degradable surfactant.

In yet another aspect, there is provided a method for analyzing a protein, the method comprising: (a) combining the protein with a solution comprising a degradable surfactant or a degradable surfactant precursor to form an isolated protein solution; (b) analyzing the isolated protein by injecting the isolated protein solution into a mass spectrometer, wherein the degradable surfactant precursor comprises a mixture of a fluorinated acid and a bifunctional linking molecule, and where the degradable surfactant comprises a compound having a fluorinated alkyl moiety and a cleavable linking moiety.

These and other aspects are described in more detail below.

### BRIEF DESCRIPTION OF THE FIGURES

FIG. 1 provides a chromatogram with an MRM of +2 ion of Cystamine ditridecafluoroheptanoic acid (CT2). Without degradation of CT2, the peak should be in excess of 10̂6 height.

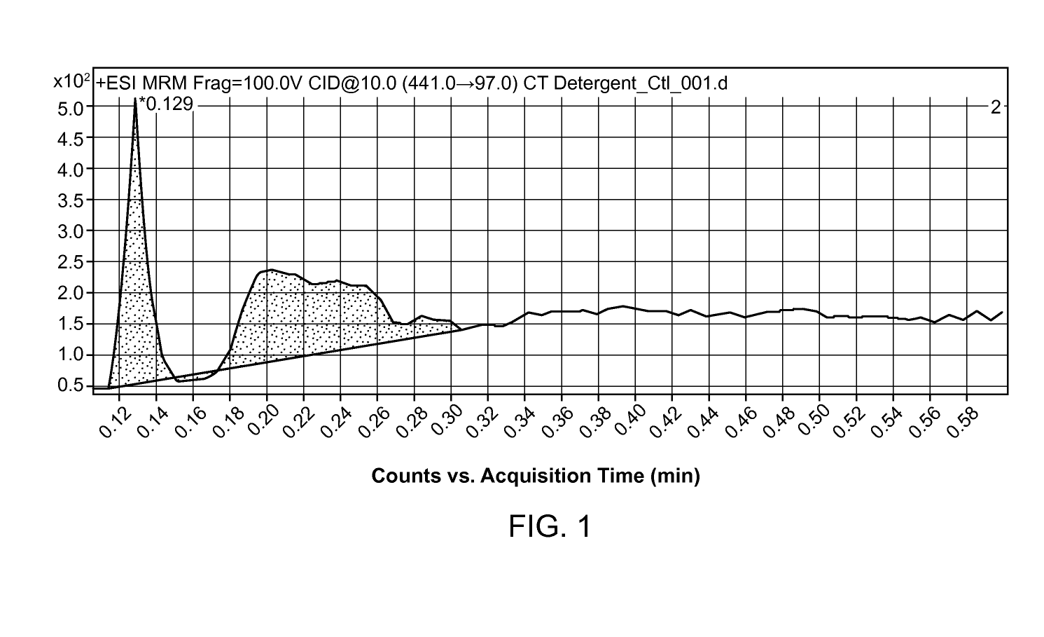

FIG. 2 provides a chromatogram of internal standards and enzymatic products from Acid Syphingomyelinase (Niemann-Pick disease), Galactocerebrosidase (Krabbe disease), beta-Glucocerebrosidase (Gaucher disease), alpha-Galactosidase (Fabry disease), and Acid alpha-Glucosidase (Pompe disease). These products and internal standards gave a range from 10̂4 to 10̂5 in peak height. The concentration of the products and internal standards is less than ppm and the concentration of the surfactant is as high as 1%.

FIG. 3 provides a chromatogram of Cystamine di-tridecafluoroheptanoic acid (MRM 2+) and Cystamine, a degraded adduct from the CT2 surfactant.

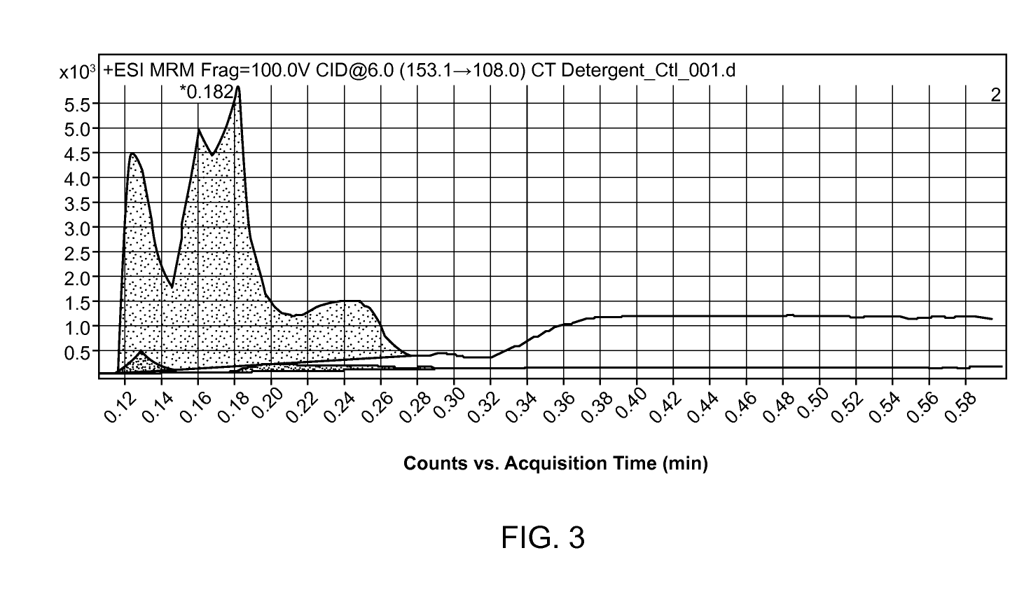

FIG. 4 provides a chromatogram of chromatogram of Cystamine, a degraded adduct from the CT2 surfactant.

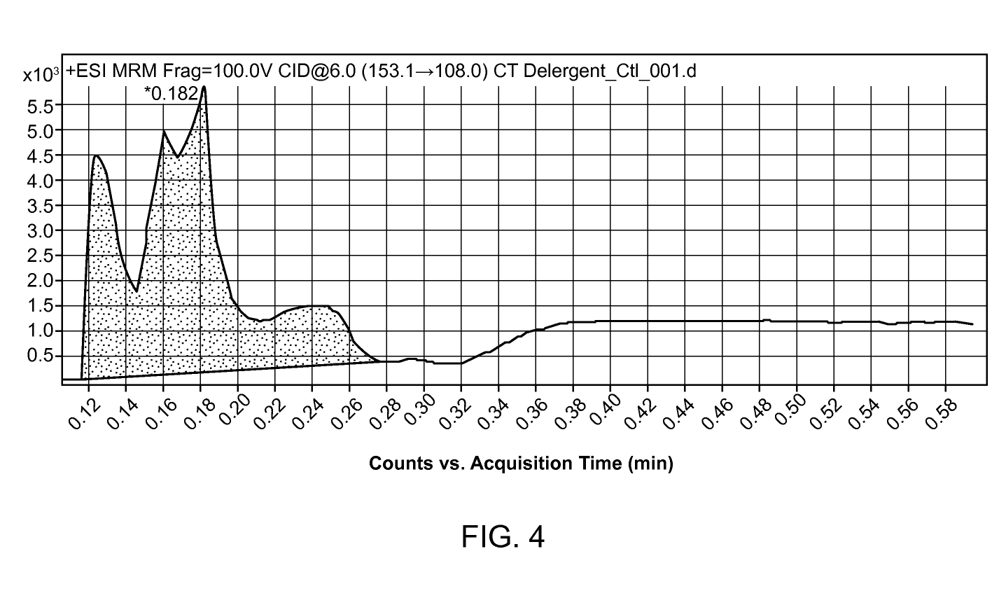

### DEFINITIONS

Unless otherwise indicated, the disclosure is not limited to specific procedures, starting materials, or the like, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a reactant” includes not only a single reactant but also a combination or mixture of two or more different reactant, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C₁-C₆ alkoxy” or “lower alkoxy” herein may, for example, may contain 1 to 3 carbon atoms, and as a further example, such substituents may contain 1 or 2 carbon atoms (i.e., methoxy and ethoxy).

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “alkylene” as used herein refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing. “Lower alkylene” refers to alkylene linkages containing from 1 to 6 carbon atoms. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

Similarly, the terms “alkenylene,” “alkynylene,” “arylene,” “aralkylene,” and “alkarylene” as used herein refer to di-radical alkenyl, alkynyl, aryl, aralkyl, and alkaryl groups, respectively.

The term “amino” is used herein to refer to the group —NZ¹Z² wherein Z¹ and Z² are hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups and the hydrocarbyl moieties C₁-C₂₄ alkyl (including C₁-C₁₈ alkyl, further including C₁-C₁₂ alkyl, and further including C₁-C₆ alkyl), C₂-C₂₄ alkenyl (including C₂-C₁₈ alkenyl, further including C₂-C₁₂ alkenyl, and further including C₂-C₆ alkenyl), C₂⁻C₂₄ alkynyl (including C₂-C₁₈ alkynyl, further including C₂-C₁₂ alkynyl, and further including C₂-C₆ alkynyl), C₅-C₃₀ aryl (including C₅-C₂₀ aryl, and further including C₅-C₁₂ aryl), and C₆-C₃₀ aralkyl (including C₆-C₂₀ aralkyl, and further including C₆-C₁₂ aralkyl). By a “functional group” is meant a group that contains one or more reactive moieites. A functional group may be a terminal substituent or may be a linking moiety. Examples of functional groups include halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O(CO)—O-alkyl), C₆-C₂₀ arylcarbonato (—O—(CO)—O-aryl), carboxy (—COOH), carboxylato (—COO—), carbamoyl (—(CO)—NH₂), mono-substituted C₁-C₂₄ alkylcarbamoyl (—(CO)—NH(C₁-C₂₄ alkyl)), di-substituted alkylcarbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N+≡C—), cyanato (—O—C≡N), isocyanato (—O—N≡C—), isothiocyanato (—S—C≡N), azido (—N≡N+═N—), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₅-C₂₀ arylamido (—NH—(CO)-aryl), imino (—CR═NH where R=hydrogen, C₁-C₂₄ alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R═hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O)₂), phosphinato (—P(O)(O—)), phospho (—PO₂), and phosphino (—PH₂), mono- and di-(C₁-C₂₄ alkyl)-substituted phosphino, and mono- and di-(C₅-C₂₀ aryl)-substituted phosphino. In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

By “linking” or “linker” as in “linking group,” “linker moiety,” etc., is meant a bivalent radical moiety. Examples of such linking groups include alkylene, alkenylene, alkynylene, arylene, alkarylene, aralkylene, and linking moieties containing functional groups including, without limitation: amido (—NH—CO—), ureylene (—NH—CO—NH—), imide (—CO—NH—CO—) , epoxy (—O—), epithio (—S—), epidioxy (—O—O—), carbonyldioxy (—O—CO—O—), alkyldioxy (—O—(CH₂)—O—), epoxyimino (—O—NH—O, epimino (—NH—), carbonyl (—CO—), carbonyloxy (—O—CO—), etc.

It will be appreciated that, unless otherwise specificied, such functional and linking groups may appear in the orientation written or in “reverse” orientation (e.g. —O—CO— or —CO—O—).

When the term “substituted” appears prior to a list of possible substituted groups, it is intended that the term apply to every member of that group. For example, the phrase “substituted alkyl and aryl” is to be interpreted as “substituted alkyl and substituted aryl.”

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

As used herein, the terms “detergent” and “surfactant” are interchangeable and equivalent, and refer to a surface active compound or material.

### DETAILED DESCRIPTION

As mentioned previously, of interest herein are materials suitable for use as detergents, as well as methods of preparing and using such materials.

### Materials

In some embodiments, the detergents of interest are cleavable detergents. By “cleavable” is meant that, under certain conditions, the detergents degrade on a molecular level into smaller components. Such conditions include elevated temperatures and/or reduced pressures, as described in more detail below (e.g., temperatures and pressures such as those commonly used in a mass spectrometer). Such conditions may also or alternatively include changes in environmental factors such as pH. Such conditions may also or alternatively include elevated temperatures that are higher than room temperature but less than the temperatures common in mass spectrometers, and reduced pressures that are lower than standard pressure but higher than the pressures common in mass spectrometers.

In some embodiments, the detergents of interest are compounds that contain one or more cleavable moieties. Cleavable moieties are functional groups that are cleaved under one or more of the conditions mentioned herein as cleaving conditions (e.g. elevated temperature, reduced pressure, changes in pH, etc.). As described in more detail below, a variety of cleavable groups are suitable, and examples include amides, disulfides, peroxides, ethers, azo groups, and the like. In some embodiments, the detergents contain two, three, or four cleavable groups. In some embodiments, the detergents contain more than four cleavable groups.

In some embodiments, the cleavable groups are positioned such that, upon cleaving, the detergents degrade into two or more constituents. In some embodiments, the constituents are volatile, such that under typical mass spectrometric temperature and pressures, the constituents are substantially converted to gases. For example, at least 75%, or at least 80%, or at least 85%, or at least 90%, or at least 95%, or at least 98%, or at least 99%, or at least 99.9%, or at least 99.99% of the constituents are converted to gases. In such embodiments, mass spectrometry may be carried out on a sample containing a degradable detergent by: (1) injecting the sample directly into the mass spectrometer and accounting for (e.g. subtracting out) the detergent's degradation constituents in the resulting spectra; (2) injecting the sample directly into the mass spectrometer and not taking measurements in the mass ranges characteristic of the degradation constituents; or (3) subjecting the sample to elevated temperature and/or reduced pressure prior to injecting the sample into the mass spectrometer in order to remove the degradation products from the detergent. In the first of these methods, the data recorded by the mass spectrometer contains information from detergent degradation components, but such information is ignored or subtracted out of the resulting spectra. In the second of these methods, the data recorded by the mass spectrometer is substantially free of information from detergent or detergent degradation components. In the last of these methods, the sample injected into the mass spectrometer has substantially no residue from the detergent, and so the data recorded by the mass spectrometer is also substantially free of information from detergent or detergent degradation components. In all three cases, spectra of the sample are obtained that are free of contamination from detergent or detergent degradation components.

The detergents of interest may, in some embodiments, be prepared from detergent preparatory components. For example, in some embodiments, the detergents of interest are prepared from two components—a first component that is a halogenated organic acid and a second component that is a difunctional base. The two components (not necessarily in a 1:1 relationship) form chemical bonds to create a degradable detergent, wherein under certain conditions, the resulting detergent is capable of degrading into smaller fragments. In some embodiments, such fragments correspond to the components that were used to prepare the detergent (i.e. the locations of bond breaking during degradation correspond to the locations of bond making during formation). In other embodiments, such fragments are substantially different than the components used to prepare the detergent (i.e., the locations of bond breaking during degradation are different from the locations of bond making during formation). In the discussion that follows, the term “preparatory components” or “synthesis components” refers to the compounds that are used to prepare the detergents of interest. Furthermore, the term “detergent degradation components” (or simply “degradation components”) refers to the species that are generated when a detergent according to the disclosure is subjected to conditions suitable to degrade the detergent (e.g. the high temperature and low pressure commonly found in a mass spectrometer).

### Halogenated Organic Acid

As mentioned above, in some embodiments the first synthesis component of the detergents of interest is a halogenated organic acid. In some such embodiments, the organic acid is a medium chain fatty acid. For example, the organic acid is a C₄-C₁₀ carboxylic acid compound, or a C₅-C₉ carboxylic acid compound. For example, the organic acid is a C₄, or C₅, or C₆, or C₇, or C₈, or C₉, or C₁₀ carboxylic acid compound. The organic acid may have a saturated linear carbon chain, or the organic acid may be substituted, branched, cyclic, unsaturated, and/or heteroatom-containing.

Variation of the organic acid (e.g. variation in carbon chain length, substitution, etc.) provides detergents with variable properties, and allows preparation of detergents tailored for specific uses. For example, isolation of some proteins is more efficient using the longer carbon chain lengths, whereas other proteins are best isolated using detergents having shorter carbon chain lengths. In some embodiments, detergents using mixtures of organic acids of various carbon chain length (including mixtures of substituted, branched, cyclic, unsaturated, and heteroatom-containing acids) may be prepared.

In some embodiments, the halogenated organic acid is a diacid, such as a dicarboxylic acid. Suitable dicarboxylic acids are C₃-C₁₂ dicarboxylic acid. It will be appreciated that a diacid as first component, when combined with a difunctional base as second component, will form a material that is dependent on the stoichiometry of components when mixed. This is analogous to a material formed by a condensation polymerization reaction. Thus, if a large excess of one of the two components is present, the molecular weight of the resulting detergent will remain low. High molecular weight detergents can be obtained by mixing the two components in equal amounts. In some embodiments, the acid may be a diacid wherein one of the acid moieties is protected by a protecting group.

In some embodiments, the acid is fluorinated, including instances where the acid is polyfluorinated. In some embodiments, the acid is perfluorinated. In some embodiments, the acid is chlorinated, including polychlorinated and perchlorinated. In some embodiments, the acid contains a mixture of halogens, such as fluorine and chlorine.

In some embodiments, the halogenated acid comprises a fluorinated alkyl moiety. For example, the fluorinated alkyl moiety has the formula —(CF₂)ₙ—CF₃, wherein n is an integer between 4 and 12.

In some embodiments, the acid has the structure R^{a}—C(═O)OH, wherein R^{a} is a halogenated alkyl moiety. For example, R^{a} has the structure —(CF₂)ₙ‘3CF₃, wherein n is an integer between 4 and 12.

As some specific examples, the first component is heptafluorobutanoic acid, nonafluoropentanoic acid, undecafluorohexanoic acid, tridecafluoroheptanoic acid, or pentadecafluorooctanoic acid, heptadecafluorononanoic acid, nonadecafluorodecanoic acid, perfluoroundecanoic acid, hexafluoroglutaric acid, perfluoroadipic acid, or tetrafluorosuccinic acid, or combinations thereof.

In some embodiments, salts or derivates (e.g. esters, amides, etc.) of the fluorinated acid may be used in preparation of the detergents of interest.

### Difunctional Base

The second synthesis component is a difunctional base. In some embodiments, the difunctional base is a diamine. In some embodiments, the diamine has two amine groups linked via a linking moiety, wherein the linking moiety is selected from alkylene, alkenylene, arylene, and alkarylene, any of which may contain one or more heteroatoms and may contain one or more substituents. For example, the linking moiety is C₁-C₁₂ alkylene, C₁-C₁₂ heteroalkylene, C₂-C₁₂ alkenylene, C₂-C₁₂ heteroalkenylene, C₅-C₁₂ arylene, C₅-C₁₂ heteroarylene, C₆-C₁₈ alkarylene, or C₆-C₁₂ heteroalkarylene. In some embodiments the linking moiety is branched, cyclic, unsaturated, heteroatom-containing, or any combination thereof.

In some embodiments, the difunctional base is a diamine containing a linking moiety as described above, wherein the linking moiety contains one or more cleavable group. Examples of cleavable groups include amides, disulfides, peroxides, ethers, azo groups, and the like. In some such embodiments, the diamine is a dimer of a monoamine, wherein the monoamine contains a dimerizable group. Suitable dimerizable groups include thiols. Accordingly, in some embodiments, the difunctional base is a dimer of an aminothiol compound (i.e. the difunctional base is a diamino disulfide compound). The dimer contains a disulfide linkage, which functions as a cleavable linkage. The dimer may be formed ahead of time and used as a dimer to prepare the detergent, or the dimer may be formed in situ (when the detergent is formed) by using the monomer and providing conditions sufficient to cause dimerization.

In some embodiments, the difunctional base is non-halogenated. In other embodiments, the difunctional base is halogenated, such as fluorinated, chlorinated, polyfluorinated, polychlorinated, perfluorinated, or perchlorinated.

In some embodiments, the difunctional base contains two amine groups selected from primary amines and secondary amines, or contains a combination of a primary amine and a secondary amine.

In some embodiments, the difunctional base has the structure given by formula FG^{b}-L^{b}-X^{b}, wherein FG^{b} is a basic group, L^{b} is a linker, and X^{b} is a dimerizable functional group. For example, FG^{b} is an amine, L^{b} is a C₁-C₁₂ alkyl or substituted alkyl, and X^{b} is a thiol or hydroxyl group.

Examples of some suitable difunctional bases include the disulfide dimerization product of any of the following aminothiols: cysteamine, 3-aminopropanethiol, 4-aminobutanethiol, 5-aminopentanethiol, 2-(methylamino)ethanethiol, 3-(methylamino)propanethiol, 4-(methylamino)butanethiol, and the like, or combinations thereof. For example, the disulfide dimerization product of cysteamine is cystamine. Similarly, the disulfide dimerization product of 3-aminopropanethiol is 3,3′-disulfanediyldipropan-1-amine, and of 3-(methylamino)propanethiol is 3,3′-disulfanediylbis(N-methylpropan-1-amine), and so on.

Further examples of suitable difunctional bases include the following: 1,2-ethylenediamine, 1,3-propanediamine, 1,4-butanediamine, and the like.

### Detergent

In some embodiments, the two synthesis components described above (i.e. halogenated organic acid and difunctional base) combine in solution to form a detergent. In some embodiments, neither the first synthesis component nor the second synthesis component alone (i.e. when not in the presence of the other synthesis component) has detergent properties. In other words, only when combined do the two synthesis components form a material with detergent properties.

The two synthesis components are used in a ratio that creates a detergent material suitable for the intended application. In some embodiments, the detergent is formed by combining the halogenated organic acid and the difunctional base in an appropriate ratio. For example, where the organic acid is a monocarboxylic acid, and the difunctional base is a diamine compound, the components may be mixed in about a 2:1 ratio of organic acid to diamine. Such a ratio would result in a detergent without an excess of either synthesis component. In some embodiments, where the difunctional base is a diamine compound having a chain length of e.g., 6 carbons or less, the detergent properties can be selected by using a suitable carboxylic acid (e.g., a fluorinated carboxylic acid having a longer chain length).

By using an excess of one synthesis component, the pH of the detergent solution can be adjusted as desired. For example, using an excess of the organic acid compared with a diamine (e.g. with a ratio of organic acid to diamine of 2.1:1, or 2.2:1, or 2.5:1, etc) affords a detergent solution with an acidic pH. Similarly, using an excess of diamine compared with an organic acid (e.g., with a ratio of organic acid to diamine of 2:1.1 or 2:1.2 or 2:1.5, etc.) affords a detergent solution with a basic pH. It will be appreciated that in the latter case, it may be necessary to titrate the diamine into the organic acid in order to ensure that any diamine present has either reacted with two organic acid molecules or no organic acid molecules.

As stated previously, combination of the two synthesis components leads to the formation of chemical bonds between such components and the formation of a detergent. Such chemical bonds may be selected from ionic bonds, covalent bonds, hydrogen bonds, or Van der Waals interactions, or a combination thereof. Bonds that have characteristics of more than one type of bonding (e.g., partial ionic and partial covalent character) are also suitable. Furthermore, combination of the synthesis components and formation of a detergent may also involve self-condensation reactions such as dimerization reactions. Such self-condensation reactions may also involve any of the bonding types mentioned above.

In some embodiments, the synthesis components combine to form a compound held together in part by ionic bonds (e.g., via an acid-base reaction). The two components bind tightly such that they cannot be separated by chromatography, but the ionic bonds are cleavable (e.g. under mass spectrometry conditions).

Also as mentioned previously, the detergents resulting from combination of synthesis components are capable of degrading (e.g., cleaving along a cleavable moiety) into degradation components under suitable conditions (also referred to herein as “degradation conditions” or “cleaving conditions”). Such degradation occurs by breaking cleavable chemical bonds present in the detergent. The cleavable chemical bonds may be the same bonds that were formed in preparation of the detergent (i.e. when synthesis components were combined and reacted with one another), or may be different from such bonds (i.e. bonds that were present in the original synthesis components). The cleavable bonds may be ionic bonds, covalent bonds, hydrogen bonds, or Van der Waals interactions. The degradation components individually have a lower molecular weight than the parent detergent, with such lower molecular weights being dependent upon the type, number, and location of cleavable moieties contained within the parent detergent.

Degradation conditions include, but are not limited to, the elevated temperatures and reduced pressures typically found in a mass spectrometer. In some embodiments, degradation conditions involve either elevated temperature or reduced pressure, but do not require both such conditions. In some embodiments, both elevated temperature and reduced pressure are required. Examples of elevated temperatures include temperatures above room temperature, such as 50° C., or 75° C., or 100° C., or 125° C., or 150° C., or 175° C., or 200° C., or 225° C., or 250° C., or 275° C., or 300° C., or greater than 300° C. Examples of reduced pressures include pressures below atmospheric pressure, such as below 0.9 atm, or below 0.8 atm, or below 0.7 atm, or below 0.6 atm, or below 0.5 atm, or below 0.4 atm, or below 0.3 atm, or below 0.2 atm, or below 0.1 atm, or below 0.05 atm, or below 0.01 atm.

In some embodiments, the degradation components are not surface active and are therefore not detergents. The degradation components are lower in molecular weight than the detergent from which they are derived, such as 50% lower, or 75% lower, or greater than 75% lower. In some embodiments the degradation components are similar or identical to the synthesis components, but in other embodiments the degradation and synthesis components are different. In some embodiments the degradation components are volatile under mass spectrometer conditions (e.g., the degradation components are substantially converted to gases as described above). In some embodiments the degradation components provide known or predictable information when analyzed by a mass spectrometer, and such information may be accounted for (e.g. subtracted out or ignored) in mass spectrometer recordings.

In some embodiments, the detergents of interest have the structure of formula (I)

R¹—(FG¹-L¹)ₘ₁—(X¹)ₘ₃-(L²—FG²)ₘ₂—R² (I)

In formula (I):

X¹ is a cleavable linkage;

m1, m2, and m3 are independently 0 or 1, provided that at least one of m1, m2, and m3 is 1;

L¹ and L² are independently selected from alkylene and substituted alkylene;

FG¹ and FG² are functional groups; and

R¹ and R² are independently selected from fluorinated alkyl moieties.

For example, in some embodiments, m1, m2, and m3 are all 1.

Also for example, in some embodiments, X¹ is selected from disulfide, azo, and peroxide linkages.

Also for example, in some embodiments, L¹ and L² are each (—CH₂)ₙ—, where n is an integer greater than 0. For example, n may be in the range 1-100, or 1-50, or 1-30, or 1-20, or 1-10. In some embodiments, L¹ and L² are fluorinated alkyl moieties. In some embodiments, L¹ and L² are selected from linear alkyl, branched alkyl, cycloalkyl, and combinations thereof.

Also for example, FG¹ and FG² are selected from carbonyloxy, amido, sulfonamide, amido, ureylene, imide, epoxy, epithio, epidioxy, carbonyldioxy, alkyldioxy, epoxyimino, epimino, carbonyl, and carbonyloxy. For example, in some embodiments, FG¹ and FG² are amido, carbonyloxy, or sulfonamide.

Also for example, R¹ and R² are each selected from polyfluoroalkyl and perfluoroalkyl moieties. For example, R¹ and R² are each —(CF₂)ₙ—CF₃, wherein n is an integer between 4 and 12. In other embodiments R¹ and R² are each branched or cyclic fluorinated alkyl.

In some embodiments, compounds having the structure of formula (I) are symmetrical. In such embodiments, m1 and m2 are the same, R¹ and R² are the same, FG¹ and FG² are the same, and L¹ and L² are the same.

In some embodiments, the detergents of interest have the structure of formula (II)

R¹—[(FG¹-L¹)ₘ₁—X¹—(L²-FG²)ₘ₂—R—]ₙ₁—(FG¹-L¹)ₘ₁—X¹—(L²-FG²)ₘ₂—R¹ (II)

In formula (II):

X¹, L¹, L², FG¹, FG², and R¹ are as defined for formula (I);

m1 and m2 are 0 or 1;

n1 is an integer equal to or greater than 0; and

R is a fluorinated alkyl linker moiety.

For example, in some embodiments, n1 is greater than 2, or greater than 3, or greater than 5, or greater than 10, or greater than 15, or greater than 25. In some embodiments, the detergent contains a mixture of compounds having the structure of formula (II) with a distribution of values of n1 (i.e., the detergent has a polydispersity index). For example, in some embodiments, n is in the range of 1-100, or in the range of 1-50, or in the range of 1-25.

Also for example, in some embodiments, R is selected from polyfluoroalkylene and perfluoroalkylene moieties. For example, R is —(CF₂)ₙ—, wherein n is an integer between 4 and 12. In other embodiments R is a branched or cyclic fluorinated alkylene moiety. In some embodiments, R and R¹ are the same except that R contains an additional linkage site (e.g. R is —(CF₂)₈— and R¹ is —(CF₂)₇—CF₃).

In some embodiments, the detergents of interest are prepared prior to use by combining the synthesis components in a predetermined ratio and under suitable conditions. In other embodiments, the detergent may be formed in situ by adding the synthesis components to the solution requiring a detergent. A combination of these methods, whereby part of the detergent is pre-formed (e.g. dimerization to form the difunctional base) and the remaining bonds are formed in situ, is also suitable using the methods and materials disclosed herein.

As mentioned previously, in some embodiments the pH of the detergent composition may be adjusted by using an excess of one of the synthesis components. In some embodiments, the pH may also be adjusted by using a separate acid or base. Acids typically used as pH adjusters may be used, including HCl, acetic acid, phosphoric acid, etc. Furthermore, bases typically used as pH adjusters may be used, including NaOH, ammonium hydroxide, etc. In some embodiments, the detergents of interest are stable and suitable for use at any pH in the ranges that are typical for the uses described herein below. For example the detergents are useful in the pH range of 1-14, or 1-7, or 7-14.

In addition to pH adjusters, other components may be used along with the detergents of interest. Such other components include solvents and solvent combinations, ionic strength modifiers, etc.

### Methods of Use

In some embodiment, the detergents of interest may be used to solubilize analytes. Examples of such uses include in the areas of proteomics, genomics, enzymatics, etc.

In some embodiments, the detergents of interest are used to solubilize proteins, such as membrane proteins and the like. In some embodiments, the detergents may be used to purify proteins, such as membrane proteins and the like. In some embodiments, the detergents may be used to assist enzymatic reactions, such as with hydrophobic targets. In some embodiments, the detergents can be used in other detergent-assisted reactions and purification methods that are known in the art, as well as in any combinations of the above-mentioned uses. During such uses, or upon completion of such methods, the solutions containing the detergent may be applied directly for analysis by mass spectrometry. No dialysis or other purification to remove the detergent is needed.

In some embodiments, the detergents of interest may be removed prior to analysis by mass spectrometry. Such removal may, for example, be effected by subjecting the solution containing the detergent to mass spectrometry conditions (e.g. high temperature and/or reduced pressure. Alternatively, in some embodiments, the detergents of interest are not removed prior to analysis by mass spectrometry, but are injected directly into the spectrometer along with the analytes of interest. Upon injection into a mass spectrometer (i.e. in a solution), the detergents of interest degrade into degradation components. Such degradation components may be siphoned off under vacuum to a waste container, or may be allowed to transit through the spectrometer along with the analyte(s) of interest. In the latter case, the mass spectrometer ion source and detectors may be powered off in the mass ranges of the degradation components such that the degradation components are not detected by the mass spectrometer. Alternatively, the ion source and detectors may be allowed to detect the degradation products, and such detection may be subtracted out of, or ignored in, the resulting spectra. In some embodiments, the detergents of interest do not cause any significant ion-suppression in the mass spectrometer.

In some embodiments, the detergents of interest are useful in applications that do not involve mass spectrometry, but require detergents nonetheless. For example, isolation and purification of biological samples for alternative analytical methods (e.g., NMR, X-ray diffraction, etc.) are suitable methods for the detergents described herein.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains.

### EXAMPLES

### Example 1

Tridecafluoroheptanoic acid and cysteamine were combined in a 1:1 molar ratio in solution to form a detergent. Based on observation, it was determined that two molecules of tridecafluoroheptanoic acid combine with two molecules of cysteamine to form a long chain of hydrophobic and hydrophilic ionic molecules, with all the characteristics of a detergent. It is believed that two molecules of cysteamine oxidize to form cystamine. Two molecules of tridecafluoroheptanoic acid then bind to the ends of cystamine to form an ionic detergent having the structure shown below:

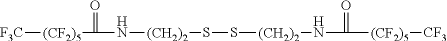

The detergent solution was titrated with either additional tridecafluoroheptanoic acid or additional cysteamine to achieve various pH values.

The resulting detergent solution was able to be diluted to any concentration suitable to dissolve membrane proteins or optimize for enzymatic reactions. For example, the enzyme Acid Syphingomyelinase (Niemann-Pick) required 0.1% detergent at pH 5.6. Another enzyme beta-Glucocerebrosidase (Gaucher) required a 1% detergent at pH 5.1.

The detergent is a 2-molecule composite (in a 2:1 stoichiometric ratio, based on tridecafluoroheptanoic acid and cystamine) that works in a wide range of pH. Before they were mixed, neither the tridecafluoroheptanoic acid nor the cystamine showed any detergent properties (i.e. no suds were observed). Once mixed, soapy suds were observed and the solution could be titrated to various pH or diluted to different concentrations.

Enzymatic reactions to detect sphingolipid metabolism disorder, requiring detergents to mimic in vivo membrane-conditions, were carried out successfully with this detergent. After the reaction, enzymatic products and the detergent were injected directly into the MS (i.e. without taking steps to remove the detergent). The detergent did not negatively affect the MS data, and it is suspected that the detergent degraded and was removed under vacuum.

### Example 2

Both trifluoroheptanoic acid and cystamine are detected by the MS/MS when their individual MRM values were input. However, at the MRM for the combined cystaminium-tridecafluoroheptanoate, no signal was detected, indicating that the detergent was degraded upon injection into the MS/MS. This experiment was repeated with many amino acids/tridecafluoroheptanoic acid complexes. Furthermore, column chromatography indicated that the combined cystaminium-tridecafluoroheptanoate was a single compound. The detergent has a low boiling point and is non-volatile, but upon exposure to the MS/MS degraded into volatile components.

It is noted that, when classical detergents are allowed into the MS, in addition to ion-suppression, clogging, etc., detergent suds are seen in the vacuum waste tubing. Suds in the waste tubing were absent with the detergents prepared herein.

### Example 3

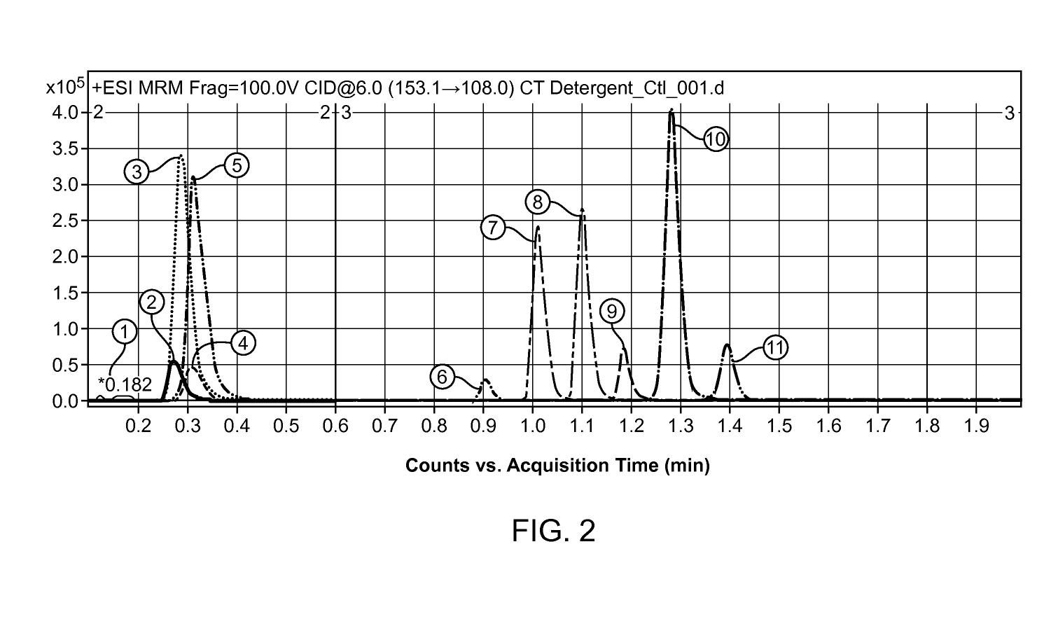

A chromatogram of internal standards and enzymatic products from Acid Syphingomyelinase (Niemann-Pick disease), Galactocerebrosidase (Krabbe disease), beta-Glucocerebrosidase (Gaucher disease), alpha-Galactosidase (Fabry disease), and Acid alpha-Glucosidase (Pompe disease) was produced (see FIG. 2). The peak IDs and retention times are shown in Table 1 including MS data for each peak by selected reaction monitoring (SRM). These products and internal standards gave a range from 10̂4 to 10̂5 in chromatogram peak height. The concentration of the products and internal standards is less than ppm and the concentration of the surfactant is as high as 1%.

TABLE 1

| Peak | | Retention | Selected Reaction |
|---|---|---|---|
| Number | Peak ID | Time | Monitoring (SRM) |
| 1 | Degraded CT2 surfactant | 0.182 | 153.1 -> 108 |
| 2 | Fabry alpha galactosidase (GLA) | 0.276 | 489.3 -> 389.2 |
| | internal standard | | |
| 3 | Fabry alpha galactosidase (GLA) | 0.278 | 484.3 -> 384.3 |
| | product | | |
| 4 | Pompe alpha-glucosidase (GAA) | 0.311 | 503.3 -> 403.2 |
| | internal standard | | |
| 5 | Pompe alpha-glucosidase (GAA) | 0.314 | 498.3 -> 398.2 |
| | product | | |
| 6 | Niemann-Pick acid | 0.905 | 370.3 -> 264.2 |
| | syphingomyelinase (ASM) | | |
| | internal standard | | |
| 7 | Niemann-Pick acid | 1.012 | 398.4 -> 264.2 |
| | syphingomyelinase (ASM) | | |
| | product | | |
| 8 | Krabbe galactocerebrosidase | 1.103 | 426.4 -> 264.2 |
| | (Gal-C) product | | |
| 9 | Krabbe galactocerebrosidase | 1.185 | 454.4 -> 264.2 |
| | (Gal-C) internal standard | | |
| 10 | Gaucher beta glucocerebrosidase | 1.283 | 482.5 -> 264.2 |
| | (ABG) product | | |
| 11 | Gaucher beta glucocerebrosidase | 1.395 | 510.5 -> 264.2 |
| | (ABG) internal standard | | |

1-4. (canceled)

5. A compound comprising at least one fluorinated alkyl moiety directly or indirectly bonded to at least one cleavable moiety, wherein the compound degrades into a plurality of degradation products upon exposure to degradation conditions.

6. The compound of claim 5, wherein the degradation conditions comprise a temperature above ambient temperature and a pressure below ambient pressure, and wherein the degradation comprises cleavage of the at least one cleavable moiety.

7. The compound of claim 6, wherein the degradation products are volatile under the degradation conditions.

8. (canceled)

9. The compound of claim 7, wherein the volatile degradation products includes fluorinated and non-fluorinated products.

10. The compound of claim 7, wherein the compound degrades into four or more volatile degradation products.

11. The compound of claim 7, wherein the compound comprises a plurality of cleavable moieties.

12. The compound of claim 11, wherein the plurality of cleavable moieties are selected from disulfides, esters, amides, and sulfonamides.

13. The compound of claim 7, wherein the surfactant comprises a plurality of fluorinated alkyl moieties.

14. The compound of claim 13, wherein the fluorinated alkyl moieties have the formula —(CF₂)ₙ—CF₃, wherein n is an integer between 4 and 12.

15. The compound of claim 13, wherein the fluorinated alkyl moieties are cycloalkyl or branched alkyl moieties.

16. The compound of claim 13, wherein the fluorinated alkyl moieties are perfluorinated.

17. The compound of claim 5, wherein the compound comprises two perfluorinated moieties separated by a linker moiety.

18. The compound of claim 17, wherein the linker moiety is non-fluorinated.

19-35. (canceled)

36. A method for preparing an analyte, the method comprising combining the analyte with a fluorinated acid and a basic compound, wherein the fluorinated acid and basic compound react to form a degradable surfactant.

37. The method of claim 36, wherein the basic compound is capable of dimerizing, and wherein the surfactant comprises two molecules of the fluorinated acid and a dimer of the basic compound.

38. The method of claim 36, wherein the surfactant degrades into volatile components upon being subjected to increased temperature and decreased pressure.

39. A method for analyzing a protein, the method comprising:
(a) combining the protein with a solution comprising a degradable surfactant or a degradable surfactant precursor to form an isolated protein solution;
(b) analyzing the isolated protein by injecting the isolated protein solution into a mass spectrometer,
wherein the degradable surfactant precursor comprises a mixture of a fluorinated acid and a bifunctional linking molecule, and where the degradable surfactant comprises a compound having a fluorinated alkyl moiety and a cleavable linking moiety.

40. A compound of claim 5, having the structure of formula (I)
R¹—(FG¹-L¹)ₘ₁—(X¹)ₘ₃—(L²-FG²)ₘ₂—R² (I)
wherein:
X¹ is a cleavable linkage;
m1, m2, and m3 are independently 0 or 1, provided that at least one of m1, m2, and m3 is 1;
L¹ and L² are independently selected from alkylene and substituted alkylene;
FG¹ and FG² are functional groups; and
R¹ and R² are independently selected from fluorinated alkyl moieties.

41. The compound of claim 40, wherein:
X¹ is a disulfide or peroxide linkage;
L¹ and L² are (—CH₂)ₙ—, where n is an integer greater than 0;
FG¹ and FG² are selected from carbonyloxy, amido, sulfonamido. amido, ureylene, imide, epoxy, epithio, epidioxy, carbonyldioxy, alkyldioxy, epoxyimino, epimino, carbonyl, and carbonyloxy; and
R¹ and R² are the same and are perfluoroalkyl moieties.

42. A compound of claim 5, having the structure of formula (II)
R¹—[(FG¹-L¹)ₘ₁—X¹—(L²-FG²)ₘ₂—R—]ₙ₁—(FG¹-L¹)ₘ₁—X¹—(L²-FG²)ₘ₂—R¹ (II)
wherein:
X¹ is a cleavable linkage;
m1 and m2 are 0 or 1;
n1 is an integer equal to or greater than 0;
L¹ and L² are independently selected from alkylene and substituted alkylene;
FG¹ and FG² are functional groups;
R is a fluorinated alkyl linker moiety; and
R¹ is a fluorinated alkyl moiety.
